(19) United States
(12) Patent Application Publication
(10) Pub. No.: US 2010/0198013 A1
(43) Pub. Date: Aug. 5, 2010

(54) STERILE ENDOSCOPIC INSTRUMENT HOUSING

(76) Inventor: Kenneth Binmoeller, Rancho Santa Fe, CA (US)

Correspondence Address: WALTER A. HACKLER, 2372 S.E. BRISTOL, SUITE B, NEWPORT BEACH, CA, 92660-0755, US

(21) Appl. No.: 12/594,489

(22) PCT Filed: Aug. 11, 2005
(86) PCT No.: PCT/US05/28486

§ 371 Date: Apr. 20, 2010

Related U.S. Application Data
(60) Provisional application No. 60/602,269, filed on Aug. 17, 2004.

(51) Int. Cl. A61B 1/00

(52) U.S. Cl. 600/121

(57) ABSTRACT

A housing is described to introduce an endosurgical or endoscopic instrument to a surgical site in a sterile manner with a sleeve that is filled with disinfecting fluid into which a the instrument can be placed and the sleeve has a membrane at one end that can be punctured by the instrument.

### PRIORITY INFORMATION

This application claims priority to U.S. Provisional Application No. 60/602,269 filed on Aug. 17, 2004, the entire contents of which are hereby expressly incorporated by reference.

### FIELD OF THE INVENTION

The device and method described herein relate in general to the field of endoscopic and endosurgical instruments, and more particularly, to such instruments for use in invasive surgical procedures on tissue in non-sterile environments.

### BACKGROUND OF THE INVENTION

There are many instances when physicians need to puncture or cut into the tissue of the body to obtain biopsies or to perform invasive surgical procedures. When these interventions are made from outside the body, the skin is disinfected prior to the intervention so that the sterile needle or surgical instrument remains sterile upon entry into the tissue. Local swabbing with solutions such as Betadyne or other disinfectants can adequately disinfect the outside surface of the tissue and kill pathogenic organisms. Once the surface is disinfected, sterile instruments that pass into the body remain sterile and the tissue usually does not become infected.

However, local disinfection is difficult when these same procedures are performed from inside the body such as through the gastrointestinal (GI) tract using an endoscope. The biopsy needle or surgical instrument is contaminated during passage through the non sterile working channel of the endoscope and further contaminated after exposure to the non-sterile environment of the gastrointestinal tract. Sometimes the patient is asked to swallow an oral disinfectant prior to the procedure to disinfect the GI tract, but this requires the ingestion of large amounts of disinfectant with potentially toxic side effects. Furthermore, oral disinfectant may not adequately disinfect all the hundreds of folds of tissue along the convoluted surfaces of the stomach and intestines.

The tissue of the GI tract is an effective barrier against the transmittance of the bacteria inside the tract to parts of the body on the other side of the tissue wall. When invasive surgeries are performed through an endoscope in the GI tract, small instruments that fit through the working channel of the endoscope are used to access lesions or target sites within or outside the GI tract. When the mucosal wall is penetrated there is the risk of infection to the patient if the surgical site is non-sterile because bacteria and other organisms can be drawn into sterile parts of the body by the instrument at the site of intervention.

Fine needle aspiration (FNA) is one example of an invasive procedure that is performed through the gastrointestinal tract. The needle is inserted through the working channel of a special endoscope equipped with an ultrasound transducer (echoendoscope) and guided into the target lesion within or outside the gastrointestinal wall. The needle, contaminated by organisms in the endoscopic working channel and on the surface of the gastrointestinal tract, can infect the target tissue. The risk of infection is particularly high for lesions that are fluid filled. When a FNA or other procedure is performed on this type of lesion, the liquid contents of the lesion can become infected and result in the formation of an abscess.

There is therefore a need for an improved device and method of performing endoscopic surgery whereby the instruments utilized can be delivered sterile to the surgical site and the surgical site itself can be generally disinfected prior to the introduction of the endoscopic instrument into the target tissue.

### SUMMARY OF THE INVENTION

A housing for delivering an endosurgical or endoscopic instrument to a surgical site in a sterile manner is described and includes a fluid sleeve suitable for housing the instrument wherein the sleeve can be filled with a disinfecting fluid. The distal end of the sleeve comprises a closed and thin membrane that can be punctured by the instrument prior to introduction into the target tissue at the surgical site.

In one embodiment the disinfecting fluid inside the sleeve can be pressurized and the membrane may have at least one fluid pathway that permits the disinfecting fluid to be released, specifically to spray out of the membrane when the fluid is pressurized. In one embodiment of the membrane the fluid pathways are formed in the membrane and in other embodiments the fluid pathway is cut into the membrane after the membrane is formed. In another aspect of the invention the fluid pathway is designed so that it causes the disinfecting fluid to exit the membrane in a defined spray pattern to disinfect the tissue at the target surgical site. In another aspect of the invention the membrane can seal around the instrument once the instrument punctures it and is advanced from the membrane into the adjacent body tissue. And in another embodiment the membrane can re-seal itself after the instrument is retracted back into the housing upon completion of the intervention.

In another aspect a system is described to introduce an endosurgical or endoscopic instrument to a surgical site in a sterile manner and includes an elongated housing comprising a sleeve into which the instrument can be introduced, the sleeve can be filled with a disinfecting fluid and comprises a membrane at one end that can be punctured by the instrument.

In another aspect, a method is described to introduce an endoscopic or endosurgical instrument to a surgical site wherein in the body includes positioning an elongated housing comprising a sleeve at the surgical site; the housing has a puncturable membrane at the distal end. The method also includes filling the sleeve with disinfecting fluid, inserting the instrument into the fluid filled sleeve from the proximal end, puncturing the membrane with the instrument and advancing the instrument to the surgical site.

In another aspect of the method the membrane located at the distal end of the housing comprises at least one fluid pathway through the membrane so that when the disinfecting fluid in the sleeve is pressurized the disinfecting fluid can be released and specifically sprayed out from the fluid pathway to disinfect the site. And in another aspect the surgical site is sprayed with the disinfectant prior to the advancement of the instrument through the membrane so that the site can be disinfected.

The details of the present invention, both as to its structure and operation, can best be understood in reference to the accompanying drawings, in which like reference numerals refer to like parts, and in which:

### BRIEF DESCRIPTION OF THE DRAWINGS

FIG. 1 is a perspective view of the endoscopic instrument housing apparatus;

FIG. 2 is a section view of the instrument housing apparatus showing the distal tip and fluid pathways in the membrane;

FIG. 3 is a section view of the distal end of the instrument housing apparatus;

FIG. 4 is a section view of the instrument housing apparatus showing the tip of the housing near the target surgical site with an instrument inside the housing;

FIG. 5 is a section view of the instrument housing apparatus showing the tip near the target site, the instrument inside the housing and the pressurized disinfectant fluid being sprayed onto the target; and

FIG. 6 is a section view of the instrument housing apparatus showing the tip of the housing against the target tissue site and the instrument extended into the tissue.

### DETAILED DESCRIPTION OF THE PREFERRED EMBODIMENT

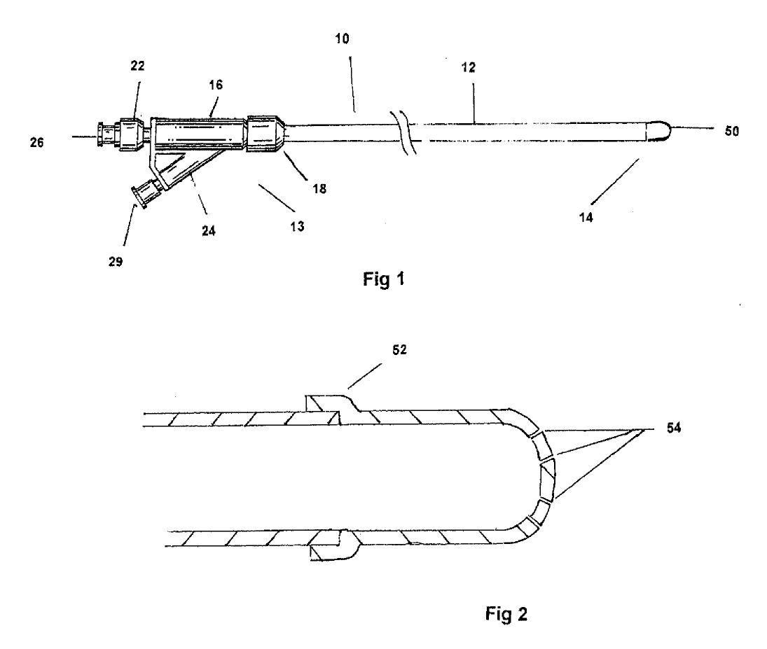

The systems, devices and methods described herein enable the creation of a disinfected or sterile environment in order to perform endoscopic procedures. FIGS. 1-6 depict an embodiment of an endoscopy housing apparatus useful for maintaining sterility of endoscopic or endosurgical instruments while these instruments are transported to the target surgical site. Also described are a device and method to disinfect the site prior to advancement of the instrument into the tissue. Also described is a method to retract the instrument into the housing upon completion of the procedure and at least partially disinfect the instrument prior to using the instrument for an additional intervention without having to withdraw the entire instrument from the endoscope.

As seen in FIG. 1, the housing 10 consists of an elongated sleeve 12, the outside diameter of the sleeve is preferably sized for placement through the working lumen of an endoscope. However the sleeve 12 can be made of various sizes and the housing can be used with or without the aid of an endoscope. In a specific embodiment, the sleeve 12 comprises a flexible material and the housing 10 comprises a proximal end 13 and a distal end 14. The proximal end 13 provided with comprises a connector 16 attached to the sleeve 12 at junction 18. The connector 16 comprises an inner lumen and is in fluid communication with the inner lumen of the sleeve 12. The connector 16 and the sleeve 12 can be joined by suitable means known in the art such as by compression fit, chemical, adhesive or heat bonding. The connector 16 comprises two arms located at its proximal end, a through arm 22 and a side arm 24. The through arm 22 comprises an end 26 through which an endoscopic instrument can be inserted into the connector. The end 26 with a seal (not shown) permits instruments to be inserted into the connector 16 while still maintaining a fluid tight condition. The side arm 24 comprises end 29 is designed to be compatible with various irrigation connections such as luer connectors, syringe connectors or threads. The side arm 24 comprises an inner lumen that is in fluid connection with the inner lumens of the through arm 22 and the sleeve 12.

The housing 14 comprises a membrane 50 positioned at the distal end of the sleeve 12 indicated by junction 52. In a specific embodiment, the membrane comprises a thin polymeric material, that may be bonded or press fitted or coupled to the sleeve 12, that can be easily punctured by the instrument. The membrane 50 is designed to provide a fluid tight seal at the end of the sleeve 12. Suitable materials for this membrane are silicone, latex, polyurethane or polyethylene although any thin walled flexible material would be adequate. The membrane is shown attached to the outside of the sleeve 12 but could be integrally formed with the sleeve 12, butt welded to the end of the sleeve 12 or attached to the inside of sleeve 12.

The membrane 50 is shown in greater detail in FIG. 2. In this figure the membrane 50 is shown with fluid pathways 54 that permit disinfectant fluid to be released and, specifically, to spray out of the pathways when the disinfecting fluid in the sleeve is under pressure. Although two pathways are illustrated in this figure, this is not meant to be limiting and a single pathway or more than one pathway may also function adequately. The fluid pathways are preferably slits in the membrane as shown in FIG. 2 but could be small through holes as well. These fluid pathways 54 are small enough to remain fluid tight when fluid inside the sleeve 12 is at atmospheric pressure. However, these fluid pathways 54 cause the fluid to exit the membrane in a spray pattern when fluid inside the inner lumen of the sleeve 12 is pressurized to a point above atmospheric pressure. When the fluid pathways 54 at the end of the membrane are used to spray disinfecting fluid on nearby tissue, the slits open up as a result of an increased fluid pressure in the sleeve 12. In a similar manner, these pathways are designed to close when the fluid pressure is reduced to prevent body fluids that may surround the outside of the housing 10, from entering the distal end 14 of the sleeve. The closure of the fluid pathways 54 may be important in order to preserve the sterile environment inside the sleeve 12 or to prevent unwanted leakage of the disinfecting fluid out of the distal end 14 of the housing.

These fluid pathways 54 can be integrally formed in the membrane 50 or can be formed in secondary operations before or after the membrane is coupled to the sleeve 12. The fluid pathways 54 can be randomly placed in which an unpredictable release or spray pattern would result once the fluid inside the housing was pressurized or the fluid pathways 54 can be formed in such a way that a particular release of spray pattern results. In one embodiment the membrane 50 may be manufactured with fluid pathways 54 that provide pre-determined release or spray patterns. For example one fluid pathway pattern may cause a narrow stream of disinfecting fluid to exit the housing end and another fluid pathway pattern may cause a broad stream of disinfecting fluid to exit the housing. In this situation, the operator could choose the housing with a particular release or spray pattern depending on the physiology of the target surgical site or the type of intervention planned.

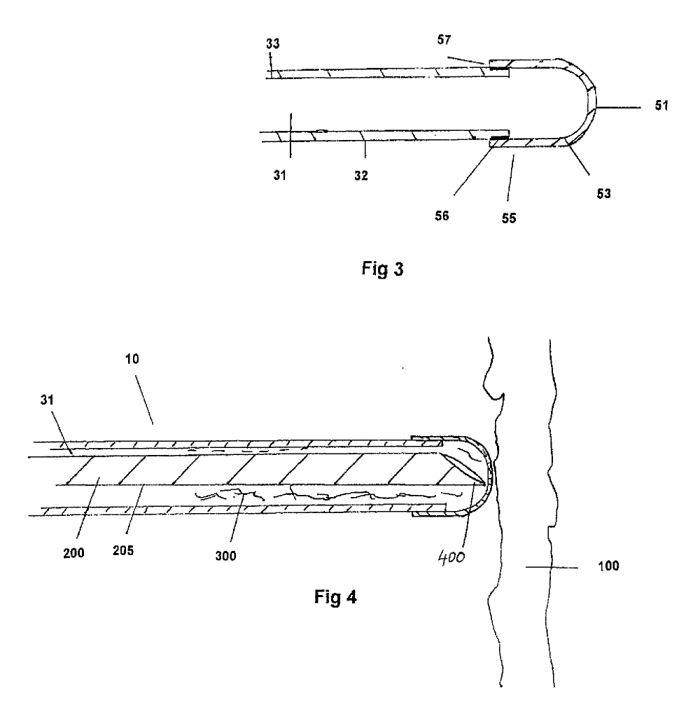

A cross section of the sleeve 12 and membrane 50 is shown in a specific embodiment in FIG. 3. The sleeve 12 comprises a luminal space 31, an outer wall 32, and an inner wall 33. Likewise the membrane has an outer wall 51 and an inner wall 53. It is important to maximize the open lumen space 31 area of the sleeve to provide maximum cross sectional diameter for instruments. It may also be important to provide enough clearance between the instrument and the inner wall 33 of the sleeve to insure that adequate fluid can cover all the outer surface area of the instrument. The sleeve 12 may be preferably sized to be placed through the working channel of an endoscope. However a larger diameter sleeve 12 may be advantageous to accommodate larger surgical instruments. If placed through an endoscope, the endoscope is particularly useful to direct the sleeve to the intended location. However a stand alone sleeve 12 that can be steered to the intended site without the aid of an endoscope is anticipated.

At the junction 55 of the membrane and sleeve 52, adhesive 56 may be placed in the gap 57 to join the two materials together. In another embodiment the membrane 50 may be integrally formed with the sleeve 12 or the membrane 50 could be press fit or butt welded to the sleeve 12. Other joining methods can also be utilized such as ultrasonic welding, friction fit or heat bonding. Any one of a number of coupling methods or designs could be used as is known to those skilled in the art.

A specific embodiment shown in FIG. 4 depicts the housing 10 in close proximity to tissue 100. This represents the condition of the housing 10 as the tip 400 is presented at the site of the intended surgical intervention. Disinfecting fluid 300 is injected into the housing by attaching a syringe to the side arm end 29. The inner lumens of the side arm 24, through arm 22 and sleeve 12 are filled with disinfecting fluid 300. An instrument 200 is presented at the through arm seal (not shown) and the seal is parted to permit introduction of the instrument 200 into the inner lumen of the through arm seal and into the inner lumen 31 of the sleeve 12. The instrument 200 is advanced to the inner wall 53 of the membrane 50. The instrument is surrounded by disinfecting fluid 300 in the space between the instrument outer edge 205 and the inner wall 33 of the sleeve 12. The fluid 300 is preferably kept under slight positive pressure.

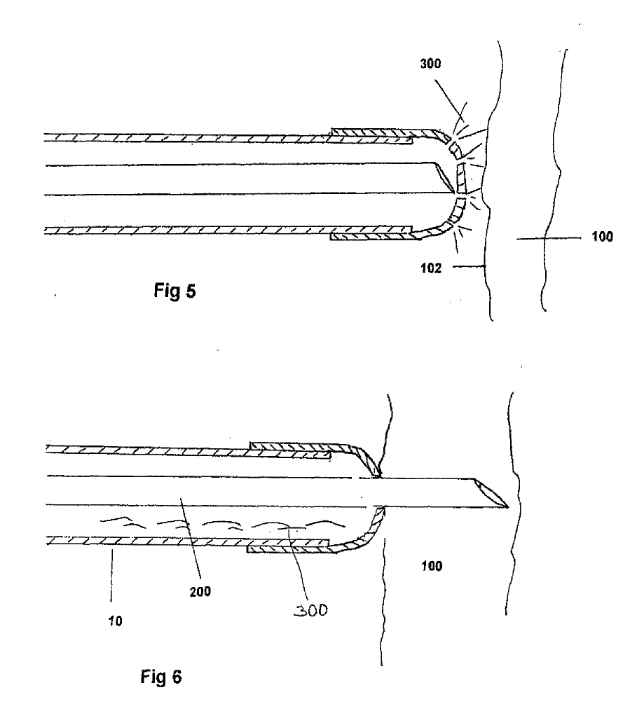

As shown in a specific embodiment in FIG. 5, increased positive pressure on the fluid causes the slits 54 to open and the disinfecting fluid 300 to be released and, specifically, to spray out onto the tissue 100. This action is intended to bathe the tissue walls 102 with disinfecting fluid 300 and generally disinfect the tissue surface 102 before introduction of the instrument. This may be useful to spray the intended tissue puncture site so as to at least partially disinfect the surface area of the tissue 100 prior to advancing the instrument. Likewise a larger area surrounding the surgical site may also be sprayed to further insure that the site is disinfected.

As shown in a specific embodiment in FIG. 6, the housing 10 is preferably kept in direct contact with the tissue wall 102 during the procedure to prevent re-contamination of the surgical site by surrounding body fluids. The instrument 200 advanced by the operator punctures the thin membrane. This procedure is particularly useful when the instrument comprises a biopsy needle that has a sharp point. Continued advancement of the instrument pushes the instrument 200 through the membrane 50 and into the tissue 100. The hole in the membrane 50 created by the instrument 200 is relatively small and preferably will seal around the body of the instrument 200 as it passes through the membrane 50. At the completion of the surgical procedure, the instrument 200 can be withdrawn into the sleeve 12. As the instrument 200 is withdrawn into the sleeve 12, the disinfecting fluid 300 will wash the instrument 200 as it is withdrawn from the tissue 100 and retracted back into the housing 10. As the instrument 200 is withdrawn completely into the housing 10, the hole created in the membrane 50 closes to provide a sterile environment for the instrument. The instrument 200 is again bathed in disinfecting fluid 300 inside the sleeve 12 and disinfected.

Once the instrument is inside the sterile housing it is at least partially sterilized. The instrument is now prepared for a possible secondary operation. This feature of the invention may be important because it allows the operator to perform multiple procedures with a single instrument and equally important it does not require the instrument to be removed and replaced by a sterile one. If the same instrument 200 can be used for multiple interventions without requiring removal of the instrument from the endoscope or even the removal of the endoscope itself, this may result in faster procedure times and reduced risk of trauma and infection to the patient.

While the particular inventions as herein shown and described in detail are fully capable of attaining the above-described objects of the invention, it is to be understood that it is the presently preferred embodiment of the present invention and is thus representative of the subject matter which is broadly contemplated by the present invention, that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more”. All structural and functional equivalents to the elements of the above-described preferred embodiment that are known or later come to be known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims.

I claim:

1. A housing for delivering an endosurgical or an endoscopic instrument to a surgical site in a sterile manner comprising:
a fluid sleeve suitable for housing said instrument, the sleeve fillable with a disinfecting fluid and having a proximal end and a distal end, and
said distal end having a membrane that can be punctured with the surgical instrument.

2. The housing of claim 1, wherein the disinfecting fluid can be pressurized inside the sleeve.

3. The housing of claim 1, wherein the membrane has at least one fluid pathway that permits the disinfecting fluid to be released from the membrane when the fluid is pressurized.

4. The membrane of claim 3, wherein the at least one fluid pathway is integrally formed in the membrane.

5. The membrane of claim 3, wherein the at least one fluid pathway is cut into the membrane after the membrane is formed.

6. The membrane of claim 3, wherein the at least one fluid pathway is configured so that the fluid sprays from the membrane.

7. The membrane of claim 6, wherein the at least one fluid pathway causes the fluid to spray from the membrane in a defined pattern.

8. The membrane of claim 1, wherein the membrane seals around the instrument once it is punctured by the instrument.

9. The membrane of claim 8, wherein the membrane self seals after the instrument is retracted into the housing.

10. The endosurgical instrument of claim 1, wherein the instrument comprises a needle.

11. A system to introduce an endosurgical or endoscopic instrument to a surgical site in a sterile manner comprising:
a housing comprising a sleeve having a proximal and distal end, into which the instrument can be introduced
and the distal end having a membrane that can be punctured by the instrument;
and the sleeve being filled with disinfecting fluid.

12. The sleeve of claim 11, wherein the disinfecting fluid is pressurized.

13. The sleeve of claim 12, wherein the membrane has at least one fluid pathway configured so that fluid sprays from the end of the membrane.

14. The membrane of claim 13, wherein the at least one fluid pathway is configured to cause the spray to have a defined pattern.

15. A method for introducing an endoscopic or endosurgical instrument to a surgical site in the body comprising:
positioning a housing comprising a sleeve at the surgical site, the housing having a puncturable membrane at the distal end; and
filling the sleeve with disinfecting fluid.

16. The method of claim 15, further comprising
inserting the instrument into the fluid-filled sleeve;
puncturing the membrane with the instrument; and
advancing the instrument to the surgical site.

17. The method of claim 15, further comprising
pressurizing the disinfecting fluid in the sleeve; and
releasing the disinfecting fluid from the membrane to disinfect the surgical site prior to advancing the instrument into the tissue at the surgical site.

18. The method of claim 16, further comprising
retracting the instrument into the fluid-filled sleeve.

19. The method of claim 17, wherein the membrane comprises at least one fluid pathway.

20. The method of claim 17, wherein releasing the disinfecting fluid includes spraying of the fluid.
